(19) United States
(12) Patent Application Publication
(10) Pub. No.: US 2017/0368026 A1
(43) Pub. Date: Dec. 28, 2017

(54) PROMOTION OF HEALING OF INTESTINAL MUCOSA USING PROLINE, SERINE AND THREONINE

(71) Applicant: NESTEC S.A., Vevey (CH)

(72) Inventors: Magali Faure, Forel (CH); Stephanie Blum-Sperisen, Pully (CH); Mohamed Nabil Bosco, Epalinges (CH); Viral Brahmbhatt, Guragon, Haryana (IN); Denis Breuille, Lausanne (CH)

(21) Appl. No.: 15/544,066

(22) PCT Filed: Jan. 22, 2016
(86) PCT No.: PCT/EP2016/051287

§ 371 Date: Jul. 17, 2017

(30) Foreign Application Priority Data
Jan. 23, 2015 (EP) 15152321.4

(51) Int. Cl. A61K 31/401; A23L 33/175; A61K 31/198

(52) U.S. Cl. CPC A61K 31/401; A61K 31/198; A23L 33/175; A23V 2002/00

(57) ABSTRACT

Methods and compositions are provided that use proline, serine and threonine to promote healing of the intestinal mucosa. A therapeutically effective amount of proline, serine and threonine is administered to an individual in need thereof, for example a human or other mammal, such as an individual that has damaged intestinal mucosa. The methods and compositions can promote mucosal healing, restore gut barrier function, repair intestinal epithelial cells, promote protection of the colonic epithelium, and/or promote the replenishment of goblet cells in the intestinal and colonic mucosa.

### BACKGROUND

The present disclosure generally relates to health and nutrition. More specifically, the present disclosure relates to methods and compositions of promoting intestinal mucosa healing using proline, serine and threonine.

The mucosa is the innermost layer of the gastrointestinal tract and surrounds the lumen that passes through the gastrointestinal tract. The gastrointestinal mucosa is formed by the innermost layer, namely the epithelium; a thin layer of smooth muscle that surrounds the epithelium, namely the muscularis mucosae; and a layer of connective tissue between the epithelium and the muscularis mucosae, namely the lamina propria.

Inflammatory bowel diseases (IBD), including ulcerative colitis and Crohn's disease, are characterized not only by the mucosal inflammation but also by the severe damage of the intestinal bather function. Recent clinical studies have featured “mucosal healing” as the most significant prognostic factor for long-term remission in IBD patients and low risk of surgical treatment in CD patients.

Clinical mucosal healing has been defined as complete repair of the epithelial layer, at both endoscopic and microscopic level. Although now recognized that epithelial lining and barrier repair is important for achieving an effective IBD treatment, there is no effective therapeutic solution available to promote mucosal healing in IBD patients. Further in this regard, mucosal healing decreases the relapse risk in patients with inflammatory bowel disease, but the role of dietary supplementation in this process has been poorly investigated.

### SUMMARY

The present disclosure provides methods for promoting intestinal mucosa healing comprising administering proline, serine and threonine to an individual in need thereof, for example an individual with an inflammatory bowel disease. The inventors found that supplementation of the diet in a rat model with specific amino acids, namely proline, serine and threonine, surprisingly repaired intestinal epithelial cells and promoted the protection of the colonic epithelium, both of which are critical steps for promoting mucosal healing. Specifically, these surprisingly beneficial therapeutic effects were achieved by administration of threonine in a dose about 4 times the theoretical threonine requirements of healthy rats, serine in a dose about 3 times the basal intake of healthy rats, and proline in a dose about 4 times the basal intake defined of healthy rats.

Accordingly, in a general embodiment, a method of promoting healing of intestinal mucosa is provided. The method comprises administering to an individual in need thereof a composition comprising a therapeutically effective amount of a combination of proline, serine and threonine.

In an embodiment, the composition is administered in an amount whereby the composition provides a dose of about 0.07 to about 0.3 g of the proline/kg body weight/day.

In an embodiment, the composition is administered in an amount whereby the composition provides a dose of about 0.07 to about 0.35 g of the serine/kg of body weight of the individual per day.

In an embodiment, the composition is administered in an amount that provides a dose of about 0.04 to about 0.20 g of the threonine/kg body weight/day.

In an embodiment, at least a portion of at least one of the proline, the serine or the threonine is a free amino acid.

In an embodiment, the individual has damaged intestinal mucosa.

In another embodiment, a method of restoring gut bather function is provided. The method comprises administering to an individual in need thereof a composition comprising a therapeutically effective amount of proline, serine and threonine. In an embodiment, the composition is administered in an amount such that the composition provides a dose selected from the group consisting of (i) about 0.07 to about 0.3 g of the proline/kg body weight/day, (ii) about 0.07 to about 0.35 g of the serine/kg of body weight of the individual per day, (iii) about 0.04 to about 0.20 g of the threonine/kg body weight/day, and (iv) combinations thereof. In an embodiment, the individual has impaired gut bather function.

In another embodiment, a method of repairing intestinal epithelial cells is provided. The method comprises administering to an individual in need thereof a composition comprising a therapeutically effective amount of a combination of proline, serine and threonine. In an embodiment, the composition is administered in an amount such that the composition provides a dose selected from the group consisting of (i) about 0.07 to about 0.3 g of proline/kg body weight/day, (ii) about 0.07 to about 0.35 g of serine/kg of body weight of the individual per day, (iii) about 0.04 to about 0.20 g of threonine/kg body weight/day, and (iv) combinations thereof. In an embodiment, the individual has damaged intestinal epithelial cells.

In another embodiment, a method of promoting protection of the colonic epithelium is provided. The method comprises administering to an individual in need thereof a composition comprising a therapeutically effective amount of a combination of proline, serine and threonine. In an embodiment, the composition is administered in an amount such that the composition provides a dose selected from the group consisting of (i) about 0.07 to about 0.3 g of the proline/kg body weight/day, (ii) about 0.07 to about 0.35 g of the serine/kg of body weight of the individual per day, (iii) about 0.04 to about 0.20 g of the threonine/kg body weight/day, and (iv) combinations thereof. In an embodiment, the individual has damaged colonic epithelium.

In another embodiment, a method of promoting growth and/or production of goblet cells is provided. The method comprises administering to an individual in need thereof a composition comprising a therapeutically effective amount of a combination of proline, serine and threonine. In an embodiment, the composition is administered in an amount such that the composition provides a dose selected from the group consisting of (i) about 0.07 to about 0.3 g of the proline/kg body weight/day, (ii) about 0.07 to about 0.35 g of the serine/kg of body weight of the individual per day, (iii) about 0.04 to about 0.20 g of the threonine/kg body weight/day, and (iv) combinations thereof. In an embodiment, the individual has depleted goblet cells.

In yet another embodiment, a method for treating inflammatory Bowel Disease (IBD) is provided. The method comprises administering to an individual in need thereof a composition comprising a therapeutically effective amount of a combination of proline, serine and threonine. In an embodiment, the composition is administered in an amount such that the composition provides a dose selected from the group consisting of (i) about 0.07 to about 0.3 g of the proline/kg body weight/day, (ii) about 0.07 to about 0.35 g of the serine/kg of body weight of the individual per day, (iii) about 0.04 to about 0.20 g of the threonine/kg body weight/day, and (iv) combinations thereof. The IBD may be Crohn's Disease or Ulcerative Colitis.

In yet another embodiment, a method for preventing or postponing relapse in an IBD patient is provided. The method comprises administering to an individual in need thereof a composition comprising a therapeutically effective amount of a combination of proline, serine and threonine. In an embodiment, the composition is administered in an amount such that the composition provides a dose selected from the group consisting of (i) about 0.07 to about 0.3 g of the proline/kg body weight/day, (ii) about 0.07 to about 0.35 g of the serine/kg of body weight of the individual per day, (iii) about 0.04 to about 0.20 g of the threonine/kg body weight/day, and (iv) combinations thereof. The IBD may be Crohn's Disease or Ulcerative Colitis.

In another embodiment, a method of making a composition for promoting healing of intestinal mucosa is provided. The method comprises adding an amount of proline, serine and threonine therapeutically effective for promoting intestinal mucosa healing to a foodstuff to form a medical food. In an embodiment, the medical food is selected from the group consisting of a fermented milk, a yogurt, a fresh cheese, a renneted milk, a confectionery bar, breakfast cereal flakes, a breakfast cereal bar, a drink, a milk powder, a soy-based product, a non-milk fermented product, or a nutritional supplement for clinical nutrition. In an embodiment the composition may be in the form of a powder, in particular a powder for reconstitution with a liquid. In an embodiment the composition may be in the form of a liquid, for example a ready-to-drink liquid oral nutritional supplement.

In an embodiment each of the proline, serine and threonine have a form individually selected from the group consisting of (i) part of the protein, (ii) free form amino acid, and (iii) mixtures thereof.

In an embodiment, the individual is selected from the group consisting of an infant, a child, an adolescent, an adult and an elderly person. In a preferred embodiment, the individual is selected from the group consisting of an adult and elderly person.

An advantage of the present disclosure is to provide methods of promoting intestinal mucosa healing and provide compositions useful in such methods.

Another advantage of the present disclosure is to promote intestinal mucosa healing by oral administration of a therapeutic nutritional composition or medicament incorporating proline, serine and threonine.

Yet another advantage of the present disclosure is to reduce or prevent inflammation using a natural compound that promotes intestinal mucosa healing with tolerable side effects or no side effects.

Another advantage of the present disclosure is to restore gut bather function, repair intestinal epithelial cells, promote protection of the colonic epithelium, and/or promote growth and/or production of goblet cells.

Additional features and advantages are described herein, and will be apparent from, the following Detailed Description and the Figures.

### BRIEF DESCRIPTION OF THE FIGURES

FIG. 1 is a graph of experimental results (number of goblet cells per μm of colic epithelium) from the example disclosed herein. Number of goblet cells per μm of colic epithelium. Data are mean±SEM. CTRL: Control; DSS: Dextran Sulphate Sodium; ALA: Alanine; 3 AA: blend of threonine, serine and proline; μm: micrometer. Differences were assessed using a non-parametric Kruskal-Wallis analysis of variance followed by a Dunn's test. A value of p<0.05 was considered statistically significant. * vs. CTRL-ALA; [symbol] vs. DSS-ALA.

FIG. 2 is a graph of experimental results (plasma concentrations of intestinal fatty acid binding protein, iFABP) from the example disclosed herein. Plasma concentration of iFABP at day 9 and day 28. Data are mean±SEM. CTRL: Control; DSS: Dextran Sulphate Sodium; ALA: Alanine; 3 AA: blend of threonine, serine and proline. Differences were assessed using a non-parametric Kruskal-Wallis analysis of variance followed by a Dunn's test. ng: nanogram. A value of p<0.05 was considered statistically significant. * vs. CTRL-ALA; [symbol] vs. DSS-ALA.

### DETAILED DESCRIPTION

As used in this disclosure and the appended claims, the singular forms “a,” “an” and “the” include plural referents unless the context clearly dictates otherwise. Thus, for example, reference to “an amino acid” or “the amino acid” includes two or more amino acids. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the composition unless expressed otherwise. When reference is made to the pH, values correspond to pH measured at 25° C. with standard equipment.

The terms “condition” and “disorder” mean any disease, condition, symptom, or indication. As used herein, an “effective amount” is an amount that prevents a deficiency, treats a condition or disorder in an individual or, more generally, reduces symptoms, manages progression of the condition or disorder or provides a nutritional, physiological, or medical benefit to the individual.

The terms “treatment” and “treating” include any effect that results in the improvement of the condition or disorder, for example lessening, reducing, modulating, or eliminating the condition or disorder. Non-limiting examples of “treating” or “treatment of” a condition or disorder include: (1) inhibiting the condition or disorder, i.e. arresting the development of the condition or disorder or its clinical symptoms and (2) relieving the condition or disorder, i.e. causing the temporary or permanent regression of the condition or disorder or its clinical symptoms. The terms “treating” and “treatment” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms do not necessarily imply that a subject is treated until total recovery. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. “Prevention” includes reduction of risk and/or severity of a condition or disorder.

“Animal” includes, but is not limited to, mammals, which includes but is not limited to, rodents, aquatic mammals, domestic animals such as dogs and cats, and farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the terms “patient” and “individual” are understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human that can benefit from the treatment.

An animal is considered “elderly” if it has surpassed the first two thirds of the average expected lifespan in its country of origin, preferably if it has surpassed the first three quarters of the average expected lifespan in its country of origin, more preferably if it has surpassed the first four fifths of the average expected lifespan in its country of origin. An “elderly human” means a person with a chronological age of 65 years or older.

As used herein, “long term administrations” are continuous administrations (e.g. at least twice a week, preferably daily) for 6 weeks or more. “Short term administrations” are continuous administrations (e.g. at least twice a week, preferably daily) for less than 6 weeks.

The terms “food,” “food product” and “food composition” mean a product or composition that is intended for ingestion by a human and provides at least one nutrient to the human. The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Any embodiment disclosed herein can be combined with any other embodiment disclosed herein.

In an aspect of the present disclosure, a method of promoting healing of intestinal mucosa is provided. The method comprises administering a composition comprising a therapeutically effective amount of proline, serine and threonine to an individual in need thereof, such as an individual having damaged intestinal mucosa. In an embodiment, the healing is complete repair of the epithelial layer at both endoscopic and microscopic level.

In another aspect of the present disclosure, a method of restoring gut bather function is provided. The method comprises administering a therapeutically effective amount of proline, serine and threonine to an individual in need thereof, such as an individual having impaired gut barrier function.

In yet another aspect of the present disclosure, a method of repairing intestinal epithelial cells is provided. The method comprises administering a therapeutically effective amount of proline, serine and threonine to an individual in need thereof, such as an individual having damaged intestinal epithelial cells.

In an additional aspect of the present disclosure, a method of promoting protection of the colonic epithelium is provided. The method comprises administering a therapeutically effective amount of proline, serine and threonine to an individual in need thereof, such as an individual having damaged colonic epithelium.

In an additional aspect of the present disclosure, a method of promoting replenishment of goblet cells in the intestinal and colonic mucosa is provided. The method comprises administering a therapeutically effective amount of proline, serine and threonine to an individual in need thereof, such as an individual having goblet cell depletion.

In any of these embodiments, the individual can be a patient having an inflammatory bowel disease.

In an embodiment, a method for treating inflammatory Bowel Disease (IBD) is provided. The method comprises administering to an individual in need thereof a composition comprising a therapeutically effective amount of a combination of proline, serine and threonine. In another embodiment, a method for preventing or postponing relapse in an IBD patient is provided. The method comprises administering to an individual in need thereof a composition comprising a therapeutically effective amount of a combination of proline, serine and threonine. The IBD may be Crohn's Disease or Ulcerative Colitis.

The composition may be administered to humans or animals such as companion animals, pets or livestock. In an embodiment, the composition is administered in an amount to provide one or more of the following doses: about 0.07 to about 0.3 g of proline/kg body weight/day; about 0.07 to about 0.35 g of serine/kg of body weight per day; and about 0.04 to about 0.20 g of threonine/kg body weight/day. In an embodiment, the composition comprises one or more of the following amounts: about 0.06 g of proline/g protein; about 0.07 g of serine/g of protein; and 0.04 of threonine/g of protein.

The composition has beneficial effects for any age group. Preferably, the composition is intended for infants, juveniles, adults or elderly. The composition can be administered to the individual for a short-term administration or a long-term-administration. The composition can be administered by a route selected from the group consisting of oral, topical, enteral and parenteral. Preferably the composition is administered enterally, for example orally. For example, the composition can have the form of a powder, a liquid concentrate, or a ready-to-drink beverage.

In an embodiment, the composition contains an additional amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, taurine, tryptophan, tyrosine, valine, and combinations thereof. In an embodiment, the composition may contain additionally an amino acid precursor. In one embodiment the composition contains an amino acid precursor selected from the cysteine precursors cystathionine, N-acethycysteine and/or DACE. In another embodiment, one or more of these additional amino acids are absent from the composition. For example, the proline, the serine and the threonine can be the only amino acids in the composition in such an embodiment.

Each of the proline, the serine and the threonine in the composition may be in free form (i.e. a monomer) or may be part of a dipeptide, a tripeptide, or a polypeptide (e.g. a protein, which as used herein means a polypeptide having 20 or more amino acids). If protein is used to provide one or more of the proline, the serine or the threonine, the protein can be intact protein, hydrolyzed protein, partially hydrolyzed protein, or a mixture of intact and hydrolyzed proteins.

The composition may be a food product, a supplement to a food product, an animal food product, or a pharmaceutical composition. For example, the product may be a nutritional composition, a nutraceutical, a drink, a food additive or a medicament. A food additive or a medicament may be in the form of tablets, capsules, pastilles or a liquid for example. Food additives or medicaments are preferably provided as sustained release formulations, allowing a constant supply of serine for a prolonged time.

The composition may be a medical food. A medical food product is specially formulated and intended for the dietary management of diseases or medical conditions (e.g., prevent or treat diseases or undesirable medical conditions). A medical food product can provide clinical nutrition, for example fulfilling special nutritional needs of patients with a medical condition or other persons with specific nutritional needs. A medical food product can be in the form of a complete meal, part of a meal, as a food additive, or a powder for dissolution.

In an embodiment, the nutritional compositions are in a form selected from the group consisting of tablets, capsules, liquids, chewables, soft gels, sachets, powders, syrups, liquid suspensions, emulsions, solutions, or combinations thereof. In an embodiment, the nutritional compositions are oral nutritional supplements. Alternatively, the nutritional compositions may be tube feedings.

The composition can provide complete nutrition or incomplete nutrition. Complete nutrition provides types and levels of macronutrients (protein, fats and carbohydrates) and micronutrients to be sufficient to be a sole source of nutrition for the animal to which it is being administered. Patients can receive 100% of their nutritional requirements from such complete nutritional compositions. Incomplete nutrition does not provide levels of macronutrients (protein, fats and carbohydrates) or micronutrients to be sufficient to be a sole source of nutrition for the animal to which it is being administered. A partial or incomplete nutritional composition is preferably used as a nutritional supplement.

The composition is preferably selected from the group consisting of milk powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavored drinks; culinary products; soups; tablets; and syrups. Milk may be any milk obtainable from animal or plant sources and is preferably cow's milk, human milk, sheep milk, goat milk, horse milk, camel milk, rice milk or soy milk. Additionally or alternatively, milk-derived protein fractions or colostrum may be used.

The composition may comprise protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. Further, the composition may contain an organic or inorganic carrier material suitable for oral or enteral administration as well as vitamins, minerals trace elements and other micronutrients in accordance with the recommendations of government bodies.

The composition may comprise a protein source, a carbohydrate source and/or a lipid source. Any suitable protein source may be used, for example animal proteins (such as milk proteins, meat proteins and egg proteins), vegetable proteins (such as soy protein, wheat protein, rice protein, and pea protein), mixtures of free amino acids, or combinations thereof. Milk proteins, such as casein and whey, and soy proteins are particularly preferred.

If the composition includes a fat source, the fat source preferably provides 5% to 50% of the energy of the composition, preferably 10% to 40%, more preferably 20% to 30% of the energy. Vegetable fats such as soy oil, palm oil, coconut oil, safflower oil, sunflower oil, corn oil, canola oil, and lecithins are particularly suitable. Animal fats such as milk fat may be included if desired.

A source of carbohydrates may provide 20% to 80% of the energy of the composition, preferably 30% to 70% of the energy of the composition. Any suitable carbohydrate may be used, for example sucrose, lactose, glucose, fructose, corn syrup solids, maltodextrins, and mixtures thereof. Dietary fiber may also be added if desired. The dietary fiber may be from any suitable origin, including for example soy, pea, oat, pectin, guar gum, gum Arabic, fructooligosaccharides, galacto-oligosaccharides, sialyl-lactose and oligosaccharides derived from animal milks.

Suitable vitamins and minerals may be included in the composition. The presence and amounts of specific vitamins and minerals will vary depending on the intended recipient of administration.

In an embodiment, the composition further comprises one or more nucleotides, synbiotics, fish oils, non-marine sources of omega-3 fatty acids, phospholipids, phytonutrients and/or antioxidants. As used herein, a synbiotic is a combination of a prebiotic and a probiotic that synergistically improves the microflora of the intestine. Non-limiting examples of suitable fish oils include fish oils providing docosahexaenoic acid (DHA) and eicosapentaenoic acid (EPA). Non-limiting examples of suitable phytonutrients include quercetin, curcumin and limonin. Antioxidants are molecules capable of slowing or preventing the oxidation of other molecules. Non-limiting examples of suitable antioxidants include vitamin A, carotenoids, vitamin C, vitamin E, selenium, flavonoids, Lactowolfberry, Goji (wolfberry), polyphenols, lycopene, lutein, lignan, coenzyme Q10 (CoQ10), hesperidine and glutathione. Non-limiting examples of phospholipids include phosphatidylcholine, phosphatidylserine and phosphatidylethanolamine.

In another aspect of the present disclosure, a method of making a composition for promoting healing of intestinal mucosa is provided. The method comprises adding an amount of proline, serine and threonine therapeutically effective for promoting intestinal mucosa healing to a foodstuff to form a medical food. For example, the medical food can be a fermented milk, a yogurt, a fresh cheese, a renneted milk, a confectionery bar, breakfast cereal flakes, a breakfast cereal bar, a drink, a milk powder, a soy-based product, a non-milk fermented product, or a nutritional supplement for clinical nutrition.

### EXAMPLE

The following non-limiting example is illustrative of promoting intestinal mucosa healing according to the present disclosure.

### Example 1

The experimental procedures were carried out in accordance to European guidelines for the care and use of laboratory animals (Directive 2010/63/UE). 30 male Sprague-Dawley rats from Janvier (France), aged 6-8 months and weighting around 500/700 g on the day of arrival, were used for this study. Animals were individually housed in cages. During the study, they had free access to food and drinking water or a dextran sulfate sodium (DSS, MW 36-44 kDa, ICN Biomedicals) solution ad libitum. Colitis was induced by treating rats with 5% DSS from DO to D8 (acute colitis) and then 2% DSS from D9 to D28 (chronic colitis). DSS was dissolved in autoclaved water and provided ad libitum to the rats. Animals of the control group were given water not containing DSS from DO to D28.

3 groups of rats received the following treatment and diets:

Group CTRL (CTRL-ALA, n=10) received the control diet, a dry semisynthetic powder consisting of (g/kg): carbohydrates 646 (wheat starch), proteins 120 (supplied by herring meal balanced to meet all amino acid requirements), lipids 64 (groundnut oil 45, sunflower oil 10, rapeseed oil 9), agar-agar 30, mineral mix 70 (UAR 205b: CaHPO4, 30.1; KCl, 7; NaCl, 7; MgO, 0.735; MgSO4, 3.5; Fe2O3, 0.21; FeSO47H2O, 0.35) and vitamin mix 10 [UAR 200]. The control diet was isonitrogenous with other diets through supplementation with alanine (40 g/kg dry matter). The threonine, serine and proline concentrations of the control diet were 5.7, 5 and 5 g/kg (dry matter), respectively.

Group DSS control (DSS-ALA, n=10) received the control diet supplemented with alanine (40 g/kg dry matter).

Group DSS (DSS-3 AA, n=10) received the control diet supplemented with a blend of 3 AAs, i.e. threonine (15 g/kg dry matter), serine (10 g/kg dry matter) and proline (15 g/kg dry matter).

During an adaptation period of 8 d, rats from each group were fed their respective diets. From DO, they received or not DSS in their drinking water as described before. On D9 and D28 blood was collected and immediately transferred in dry tubes, centrifuged and the plasma was frozen at −20° C. until further analyses. Plasma concentrations of intestinal fatty acid binding protein (iFABP) were assessed using a commercial ELISA kit.

At the end of the experiment (D28), animals were anesthetized using a combination of ketamine and xylasine and then euthanized by cervical dislocation. As soon as animals were euthanized, an abdominal midline incision was performed and the colon was collected from the colocecal junction to the anal verge. The colon was rinsed, small pieces of colon from both the proximal, median and distal colon were collected and placed in refrigerated 4% formalin. Samples were dehydrated and then embedded in wax in order to obtain transversal sections. Colonic slides were immunolabeled with an anti-NUC2 antibody in order to assess the number of goblet cells on a determined length of the colonic mucosa.

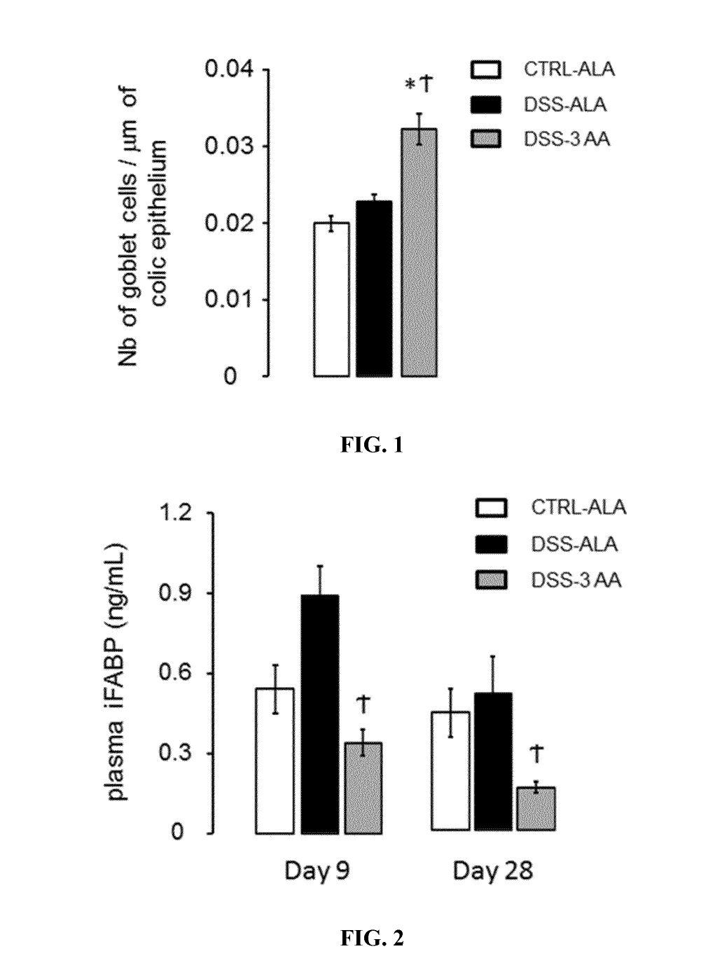

The number of goblet cells per μm of colic epithelium is presented in FIG. 1. The number of goblet cells per μm of colic epithelium was significantly higher in the DSS-3 AA group as compared to CTRL (p<0.01) and DSS groups fed iso-nitrogenous diets through supplementation of alanine. This increase in goblet cells is required to promote the protection of the colonic epithelium, as an initial step for promoting mucosal healing.

In this regard, the first line of defense of the mucosal immune system is the layer of epithelial cells, which is covered by mucus biofilm secreted from goblet cells with interspersed bacteria, mainly composed of mucins. The proportion of goblet cells among epithelial cell types increases caudally from duodenum (4%) to distal colon (16%), similar to the increasing number of microbial organisms present in the proximal intestine to colon. Goblet cells are therefore key players to ensure the intestinal and colonic mucosal protection from the constant aggressions triggered by luminal contents including for instance the microbiota. Along this line, goblet cells and their secreted products such as mucins have also been shown to inhibit apoptosis and stimulate cell migration, implying a bioactive role in maintaining the integrity of the surface epithelial layer and promoting mucosal repair and healing. Goblet cells also co-secrete trefoil factors such as TFF3, which have been shown to be involved in epithelial restitution and wound healing.

The plasma concentration of iFABP is presented in FIG. 2. On day 9, the plasma concentration of iFABP tended to increase in the DSS-ALA group as compared to CTRL-ALA (not significant). Compared to the DSS-ALA group, the plasma iFABP concentration was significantly reduced in the DSS group supplemented with the blend of 3 AA, to a concentration similar to the untreated control group. Similarly, at day 28, the plasma concentration of iFABP was significantly lower in DSS-3 AA than in the DSS-ALA group. This decrease in plasma iFABP concentration in the DSS group receiving a supplementation in the 3 AA indicates the repair of intestinal epithelial cells, which is a required process to achieve mucosal healing.

In this regard, fatty acid binding proteins (FABP) are a class of low molecular weight (14-15 kDa) cytosolic proteins found in high concentrations in tissues involved in the uptake and consumption of fatty acids. iFABP is highly expressed in cells present on the tops of the villi, the initial site of destruction in numerous intestinal diseases. iFABP is primarily limited to mature intestinal cells of the small and large intestine. It circulates in low amounts in the blood stream of healthy individuals. iFABP is a useful plasma/urinary marker for early intestinal cell death and levels rise rapidly after episodes of acute intestinal ischaemia and inflammation. The level of circulating iFABP has been reported to correlate with the histological status of the epithelium after intestinal ischaemia-reperfusion in experimental studies. An elevated serum iFABP concentration in patients with ulcerative colitis was also suggested to reflect the presence of intestinal inflammation.

Assessment of plasma iFABP is therefore recognized as the most promising endogenous intestinal cell protein marker to assess enterocyte injury, as these proteins are specifically expressed in the gut and released immediately into the circulation upon cell damage.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of promoting healing of intestinal mucosa comprising administering to an individual in need thereof a composition comprising a therapeutically effective amount of a combination of proline, serine and threonine.

2. The method of claim 1, wherein the composition is administered in an amount that provides about 0.07 to about 0.3 g of the proline/kg body weight/day.

3. The method of claim 1, wherein the composition is administered in an amount that provides about 0.07 to about 0.35 g of the serine/kg of body weight/day.

4. The method of claim 1, wherein the composition is administered in an amount that provides about 0.04 to about 0.20 g of the threonine/kg body weight/day.

5. The method of claim 1, wherein at least a portion of at least one of the proline, the serine or the threonine is a free amino acid.

6. The method of claim 1, wherein the individual has damaged intestinal mucosa.

7. A method of restoring gut barrier function comprising administering to an individual in need thereof a composition comprising a therapeutically effective amount of proline, serine and threonine.

8. The method of claim 7, wherein the composition is administered in an amount that provides a dose selected from the group consisting of (i) about 0.07 to about 0.3 g of the proline/kg body weight/day, (ii) about 0.07 to about 0.35 g of the serine/kg of body weight of the individual per day, (iii) about 0.04 to about 0.20 g of the threonine/kg body weight/day, and (iv) combinations thereof.

9. The method of claim 7, wherein the individual has impaired gut barrier function.

10. A method of achieving a result selected from the group consisting of repairing intestinal epithelial cells, promoting protection of the colonic epithelium, promoting growth and/or production of goblet cells, treating inflammatory Bowel Disease (IBD), and preventing or postponing relapse in an IBD patient comprising administering to an individual in need thereof a composition comprising a therapeutically effective amount of a combination of proline, serine and threonine.

11. The method of claim 10, wherein the composition is administered in an amount that provides a dose selected from the group consisting of (i) about 0.07 to about 0.3 g of proline/kg body weight/day, (ii) about 0.07 to about 0.35 g of serine/kg of body weight of the individual per day, (iii) about 0.04 to about 0.20 g of threonine/kg body weight/day, and (iv) combinations thereof.

12. The method of claim 10, wherein the individual has damaged intestinal epithelial cells.

13-14. (canceled)

15. The method of claim 10, wherein the individual has damaged colonic epithelium.

16-17. (canceled)

18. The method of claim 10, wherein the individual has depleted goblet cells.

19-23. (canceled)

24. The method of claim 1, wherein the composition is a medical food in a form selected from the group consisting of a nutritionally complete product, a drink, a dietary supplement, a meal replacement, a food additive, a supplement to a food product a powder for dissolution, an enteral nutrition product, an infant formula, and combinations thereof.

25. A composition for promoting healing of intestinal mucosa, the composition comprising protein, proline, serine and threonine, each of the proline, serine and threonine have a form individually selected from the group consisting of (i) part of the protein, (ii) free form amino acid, and (iii) mixtures thereof, the composition comprising an amount selected from the group consisting of (i) about 0.06 g of the proline/g of the protein; (ii) about 0.07 g of the serine/g of the protein; (iii) 0.04 of the threonine/g of the protein, and (iv) combinations thereof.
